(19) United States
(12) Patent Application Publication
(10) Pub. No.: US 2024/0138417 A1
(43) Pub. Date: May 2, 2024

(54) TRICHODERMA-BASED BIOENHANCER FOR FOLIAR APPLICATION IN PLANTS

(71) Applicant: YPF TECNOLOGÍA S.A., Ciudad Autónoma de Buenos Aires (AR)

(72) Inventors: María Cecilia ORSINI, La Plata (AR); Maria Juliana GONZALEZ, Ringuelet La Plata (AR); Sebastián REINOSO, Berisso (AR); Walter Alberto VARGAS, La Plata (AR)

(21) Appl. No.: 18/495,900

(22) Filed: Oct. 27, 2023

Related U.S. Application Data
(60) Provisional application No. 63/419,803, filed on Oct. 27, 2022.

(51) Int. Cl. A01N 63/38; A01N 39/04; A01N 41/10

(52) U.S. Cl. CPC A01N 63/38; A01N 39/04; A01N 41/10

(57) ABSTRACT

The present disclosure provides a formulation for foliar application in plants comprising an extract obtained from the culture medium of specific isolates from the Trichoderma genus. The disclosed formulation has both growth enhancing and fungicidal activity. The present disclosure also provides methods involving the disclosed formulation, as well as uses thereof.

### FIELD OF THE INVENTION

The present invention generally relates to the field of agricultural technology for enhancing the performance of industrial crops. In particular, the invention refers to a bioenhancer for foliar application in plants having both growth enhancing and fungicidal activity. More particularly, the present invention relates to a formulation comprising an extract obtained from the culture medium of specific isolates from the Trichoderma genus.

### BACKGROUND OF THE INVENTION

Crops of agronomically interesting plants are usually exposed to a great number of diseases having a strong impact on the harvest yield. The traditional way for addressing these issues relies on the use of chemical pesticides. However, said chemical pesticides decrease the quality of the environment and compromise the farmer's health. In addition to the significant direct effects of agrochemicals on the environment, their disproportionate use is another relevant aspect to be taken into account, especially considering that it promotes the developing of resistance in the targeted organisms. The consequence of this effect has a major impact since more aggressive agents must progressively be used on the pathogenic microorganisms, which in turn results in an increasing aggressiveness on the environment and the farmer.

Therefore, the development of new strategies for controlling phytopathogens that can guarantee crop health is of global interest, specially without compromising the ecological integrity of the environment while reducing their impact on human health.

Currently, national and international sectors of agribusiness have placed a considerable interest on the development of pests control products of biological origin for addressing plant diseases.

The effectiveness of Trichoderma strains and their application for protecting crop health has been known for decades in the international scientific community (Samuels & Hebbar, 2015). Several advanced genetic studies are known as well as the genomic sequences of the most industrially relevant strains (http://genome.jgi.doe.gov/; Druzhinina et al., 2011). In fact, Trichoderma spp. is the most successful biological control agent worldwide, which has shown an enormous potential, both as a fungicide and as a growth promotor, and even with different industrial applications (enzymes production, bioremediation, etc.) (Lu et al., 2004, Woo et al., 2006).

Trichoderma comprises a genus of filamentous fungi that inhabit the soil, and pose significant benefits to the biotechnological industry, ranging from the production of enzymes with industrial significance to their use for biological control of crop diseases. The use of Trichoderma in the field of agricultural industry is based on its antagonistic capacity for pathogenic agents, including other filamentous fungi. As such, Trichoderma strains may exert biological control via three combined mechanisms of action: mycoparasitism, antibiosis and niche competition (Harman et al., 2004; Druzhinina et al., 2011). The joint action of these three mechanisms gives Trichoderma a considerable advantage over chemical fungicidal agents, since it is not directed to a single point of action in pathogens, but instead displays a synergic effect at the structural, physiological and nutritional levels.

The use of Trichoderma-based biopesticides has been promoted throughout Europe, Australia and the USA. In Argentina, for example, the company Rizobacter has launched a product, the active ingredient of which is a Trichoderma harzianum isolate, which has shown effective biocontrol capacity for the most significant diseases in wheat and barley crops.

The present inventors have additionally developed a market-available bioinoculant comprising Bradyrhizobium japonicum and a particular Trichoderma harzianum strain, described in patent application US 2020/0048157 A1.

Nevertheless, there are still several difficulties to overcome in the field of biopesticides, such as finding microorganisms with specific activity against certain pathogens, as well as developing effective formulations. Additionally, both biotic and abiotic factors such as climate, pressure and competition with endogenous microflora may reduce the performance of the biopesticide agents.

Therefore, there is still a need for the development of a bioenhancer for extensive farming, which also is able to protect several agricultural crops of interest, and at the same time allows for a cost-effective, safe and easy-to-use formulation having a high commercial durability and longer viability.

### SUMMARY OF THE INVENTION

The present invention describes a formulation for foliar application in plants comprising an extract obtained from the culture medium of a strain of Trichoderma spp.

In a particular embodiment, the extract is obtained from the culture medium of a strain of Trichoderma harzianum.

In a particularly preferred embodiment, the strain of Trichoderma harzianum is the Trichoderma harzianum strain which was deposited at the ATCC on May 20, 2019, under Accession Number PTA-125914.

In another particular embodiment, the formulation for foliar application of the invention is in liquid form, and comprises 20-30 μg/ml of proteins, as determined by the Lowry assay. Preferably, the formulation comprises 25 μg/ml of proteins.

In a preferred embodiment, the liquid formulation for foliar application of the invention comprises cysteine, alanine and arginine as free amino acids. Preferably, the liquid formulation comprises 15-25 μg/100 ml cysteine, 9-13 μg/100 ml alanine and 140-160 μg/100 ml arginine.

In another preferred embodiment, the liquid formulation for foliar application of the invention comprises lysine, glutamate, isoleucine, phenylalanine, cysteine, valine, glycine, alanine, leucine, arginine and proline after being subjected to hydrolysis conditions. Preferably, the liquid formulation comprises 12-16 μg/100 ml lysine, 20-25 μg/100 ml glutamate, 25-30 μg/100 ml isoleucine, 28-33 μg/100 ml phenylalanine, 45-55 μg/100 ml cysteine, 40-50 μg/100 ml valine, 50-60 μg/100 ml glycine, 50-60 μg/100 ml alanine, 75-85 μg/100 ml leucine, 100-110 μg/100 ml arginine and 150-160 μg/100 ml proline.

In another particular embodiment the liquid formulation for foliar application of the invention comprises at least one phytohormone selected from indole acetic acid, zeatin, gibberellin (GA1), and a combination thereof. Preferably, the liquid formulation comprises indole acetic acid, zeatin and GA1. More preferably, the liquid formulation comprises 9.7 ng/ml indole acetic acid, 54.3 ng/ml zeatin and 11.3 ng/ml GA1.

It is another aspect of this invention to provide a method for producing a formulation for foliar application in plants, comprising
a) culturing a strain of Trichoderma spp in conditions such that an increase of the biomass of the Trichoderma spp strain is observed;
b) centrifuging the culture obtained in step a); and
c) separating the solids from the supernatant of the culture medium;
   thus obtaining the formulation from the supernatant of the culture medium.

In a particular embodiment of the invention, the supernatant separated in step c) of the method for producing a formulation for foliar application in plants is spray-dried to obtain a solid formulation. Preferably, a polymer is added to the supernatant separated in step c) of the method, prior to spray-drying the supernatant. More preferably, the polymer is maltodextrin. Even more preferably, the maltodextrin is added to the supernatant at a concentration of at least 10% on a dry basis, most preferably, at a concentration of 10% on a dry basis.

It is another aspect of this invention to provide a formulation for foliar application in plants which is obtained by
a) culturing a strain of Trichoderma harzianum in conditions such that an increase of the biomass of the Trichoderma harzianum strain is observed;
b) centrifuging the culture obtained in step a); and
c) separating the solid Trichoderma harzianum biomass from the supernatant of the culture medium;
   thus obtaining the formulation from the supernatant of the culture medium.

It is yet another aspect of this invention to provide a method for protecting agricultural crop plants against the infection by phytopathogenic fungi and/or promoting growth of said crop plants, comprising applying an effective mount of the formulation of the invention to the plants by means of foliar application.

In an embodiment of this aspect, the crop plants are selected from soybean, wheat, maize, sunflower, cotton, sorghum, alfalfa, flax, canola, chickpea, rice, potato, onion, yerba mate (Ilex paraguariensis), tea.

In another embodiment of this aspect, the crop plants are fruit crop plants. Preferably, the fruit crop plants are selected from the group consisting of apple tree, pear tree, citrus tree, grapevine, tomato, cucumber, eggplant, cotton and squash.

In a more preferred embodiment of this aspect of the invention, the phytopathogenic fungi may be selected, without limitation, from the group consisting of Fusarium sp, Colletotrichum sp., Cercospora sp, Sclerotinia sp., and Rhizoctonia sp.

In a particularly preferable embodiment, the formulation for foliar application is applied to the plants in combination with at least one chemical pesticidal agent. Preferably, the at least one chemical pesticidal agent is selected from at least one fungicidal agent, at least one non-hormonal herbicidal agent, and at least one hormonal herbicidal agent. More preferably, the at least one fungicidal agent is triazole, strobirulin, or a combination thereof; the at least one non-hormonal herbicidal agent is fomesafen or diclosulam; or the at least one hormonal herbicidal agent is 2,4-dichlorophenoxyacetic acid, picloram, or a combination thereof.

It is yet another aspect of this invention to provide the use of the formulation for foliar application of the invention for protecting agricultural crop plants against the infection by phytopathogenic fungi and/or promoting growth of said crop plants.

In an embodiment of this aspect, the crop plants are selected from soybean, wheat, maize, sunflower, cotton, sorghum, alfalfa, flax, canola, chickpea, rice, potato, onion, yerba mate (Ilex paraguariensis).

In another embodiment of this aspect, the crop plants are fruit crop plants. Preferably, the fruit crop plants are selected from the group consisting of apple tree, pear tree, citrus tree, grapevine, tomato, cucumber, eggplant, cotton and squash.

In a preferred embodiment of this aspect, the phytopathogenic fungi may be selected, without limitation, from the group consisting of Fusarium sp, Colletotrichum sp., Cercospora sp, Sclerotinia sp., and Rhizoctonia sp.

In a more preferred embodiment of the invention, the phytopathogenic fungi are selected from the group consisting of Fusarium tucumaniae, Colletotrichum truncatum, Cercospora sojina, and Rhizoctonia solani.

### BRIEF DESCRIPTION OF THE DRAWINGS

FIG. 1. Evaluation of the biofungicide effect of the formulation of the invention.

FIG. 2. Percentage increase of secondary metabolites after foliar application of the formulation in soybean plants without inoculation with Y-TERRA, in three different doses (1, 2 and 3 L/hectare, referred to as DOSE 1, DOSE 2 and DOSE 3, respectively).

FIG. 3. Percentage increase of secondary metabolites after foliar application of the formulation in soybean plants inoculated with Y-TERRA, in three different doses (1, 2 and 3 L/hectare, referred to as DOSE 1, DOSE 2 and DOSE 3, respectively).

FIG. 4. Percentage increase of secondary metabolites after foliar application of Mixtar FI, the formulation of the invention, and a combination thereof in soybean plants without inoculation with Y-TERRA.

FIG. 5. Crop development of soybean plants treated with Mixtar FI, the formulation of the invention, and a combination thereof, without inoculation with Y-TERRA. A) Visual evaluation. B) Aerial and radicular regions length measurements.

FIG. 6. Percentage increase of secondary metabolites after foliar application of Mixtar FI, the formulation of the invention, and a combination thereof in soybean plants inoculated with Y-TERRA.

FIG. 7. Crop development of soybean plants treated with Mixtar FI, the formulation of the invention, and a combination thereof, previously inoculated with Y-TERRA. A) Visual evaluation. The application of the formulation of the invention was assayed both with a without a spray-dried formulation, with no difference in the performance being detectable. B) Aerial and radicular regions length measurements.

FIG. 8. Field assays results for soybean crops. Yields in kg/ha of soybean crops treated with the formulation of the invention, applied at 1.5 and 3 L/ha, Howler, chemical fungicide, and the formulation of the invention in combination with chemical fungicide. Trials carried out in three different locations. A) Pergamino B) Barrow C) Monte Buey.

FIG. 9. Field assays results for soybean crops. Yields in kg/ha of soybean crops treated with the formulation of the invention, applied at 1.5 and 3 L/ha, Howler, chemical herbicide, and the formulation of the invention in combination with chemical herbicide. Trials carried out in two different locations. A) Balcarce B) Miramar

FIG. 10. Sections of lettuce hypocotyls elongation bioassay. The results are expressed as average±SD. Statistically significant differences were determined with the software GraphPad Prism 5 (**p<0.01; ***p≤0.005). The data were subjected to a variance analysis (ANOVA) with the Dunnett test, wherein the control group corresponds to hypocotyls incubated with water.

FIG. 11. Cytokinin activity bioassay by means of cucumber cotyledons greening. A) Chlorophyll content determined by the manual SPAD detector. B) Chlorophyll content determined by extraction with acetone. The results are expressed as average±SD. Statistically significant differences were determined with the software GraphPad Prism 5 (*p<0.01; ***p≤0.005). The data were subjected to a variance analysis (ANOVA) with the Dunnett test, wherein the control group corresponds to cotyledons incubated with water.

FIG. 12. Biomass-increase cytokinin activity. (A) Representative photograph of the cotyledons of Cucumis sativus after remaining 5 days in the treatments with phytohormone, distilled water and different concentrations of the formulation, and then exposed to light. B) Quantification of the fresh weight of cotyledons. The results are expressed as average±SD of an assay (n=10). Statistically significant differences were determined with the software Graph Pad Prism 5 (***p≤0.005). The data were subjected to a variance analysis (ANOVA) with the Dunnett test, wherein the control group corresponds to cotyledons incubated with water.

FIG. 13. Bioassay of root growth in A. thaliana. (A) Growth of the main root. (B) Number of side roots after 2 days from the beginning of the treatment. The results are expressed as average±SD. Statistically significant differences were determined with the software GraphPad Prism 5 (***p≤0.005). The data were subjected to a variance analysis (ANOVA) with the Dunnett test, wherein the control group corresponds to seedlings incubated with water.

### DETAILED DESCRIPTION OF THE INVENTION

The present application discloses a biological formulation for foliar application in plants, having both growth-enhancing and fungicidal activity. More particularly, said formulation has the above mentioned combined effects, and comprises a mixture of peptides obtained from the culture medium of a strain of Trichoderma spp.

During its growth, Trichoderma produces different classes of compounds, among which can be found proteins with enzymatic and other activities, oligosaccharides, as well as other low-molecular weight compounds and secondary metabolites. The present inventors have found that these compounds have fungicidal and growth-enhancing properties when applied to plants within a formulation for foliar application.

Therefore, it is as aspect of the present invention to provide a formulation for foliar application in plants, comprising an extract obtained from the culture medium of a strain of Trichoderma spp.

The term “extract obtained from the culture medium of a strain of Trichoderma spp” is to be understood as referring to a mixture of compounds generated and released to the culture medium by a particular strain of Trichoderma spp when cultured in an appropriate medium and conditions such that it experiences a proper growth in its biomass. The extract is obtained from the culture medium after separating the solids from the liquid phase of the culture, typically by centrifuging and extracting the resulting supernatant.

The extract thus obtained may be used as separated from the culture medium, that is, as a solution comprising the afore mentioned compounds generated by the Trichoderma strain (liquid form), or it may be spray-dried after the centrifugation and separation of the liquid phase of the medium culture, thus obtaining a solid formulation (solid form).

In the case that the formulation is in a solid form, a person of skill in the art will understand that the solid formulation first must be dissolved or suspended in a proper carrier for its foliar application to the target plants. It is within the expected knowledge for such a person of skill in the art to select a proper carrier.

Within the scope of the invention, several Trichoderma strains may be used for producing the extract comprised within the formulation for foliar application. Preferably, a strain of Trichoderma harzianum is used. Most preferably, the extract is obtained from the culture medium of the Trichoderma harzianum strain which was deposited at the ATCC on May 20, 2019, under Accession Number PTA-125914.

As previously mentioned, the extract comprises a series of classes of compounds produced by the Trichoderma strain, among which proteins, free amino acids and phytohormones may be found. Particularly, there are several amino acids which are known to be related to a reduction in the effects of abiotic stress of plants. A formulation comprising such amino acids is thus preferable.

In a particular embodiment, when the formulation for foliar application is in liquid form, it comprises 20-30 μg/ml of proteins, as determined by the Lowry assay. Preferably, the formulation comprises 25 μg/ml of proteins.

In another particular embodiment, the liquid formulation for foliar application of the invention comprises cysteine, alanine and arginine as free amino acids. Preferably, the liquid formulation comprises 15-25 μg/100 ml cysteine, 9-13 μg/100 ml alanine and 140-160 μg/100 ml arginine.

In another embodiment of this aspect, the liquid formulation for foliar application of the invention comprises lysine, glutamate, isoleucine, phenylalanine, cysteine, valine, glycine, alanine, leucine, arginine and proline after being subjected to hydrolysis with methanesulfonic acid (MSA). Preferably, the liquid formulation comprises 12-16 μg/100 ml lysine, 20-25 μg/100 ml glutamate, 25-30 μg/100 ml isoleucine, 28-33 μg/100 ml phenylalanine, 45-5514/100 ml cysteine, 40-50 μg/100 ml valine, 50-60 μg/100 ml glycine, 50-60 μg/100 ml alanine, 75-85 μg/100 ml leucine, 100-11014/100 ml arginine and 150-160 μg/100 ml proline.

As mentioned above, the Trichoderma strain may produce phytohormones during its growth, particularly indole acetic acid, zeatin and/or gibberellin (GA1). Correspondingly, in another particular embodiment the liquid formulation for foliar application of the invention comprises at least one phytohormone selected from indole acetic acid, zeatin, gibberellin (GA1), and a combination thereof. Preferably, the liquid formulation comprises indole acetic acid, zeatin and GA1. More preferably, the liquid formulation comprises 9.7 ng/ml indole acetic acid, 54.3 ng/ml zeatin and 11.3 ng/ml GA1.

It is also within the scope of the present invention a method to produce the formulation for foliar application described herein. As described above, the formulation comprises an extract which is obtained from the supernatant generated when a culture medium wherein a strain of Trichoderma spp was grown is centrifuged.

Therefore, the method for producing a formulation for foliar application in plants according to the invention comprises
a) culturing a strain of Trichoderma spp in conditions such that an increase of the biomass of the Trichoderma spp strain is observed;
b) centrifuging the culture obtained in step a); and
c) separating the solids from the supernatant of the culture medium;
   thus obtaining the formulation from the supernatant of the culture medium.

The term “obtaining the formulation from the supernatant of the culture medium” is to be understood in broad sense, meaning that the formulation may consist of the supernatant as obtained in step c) of the method, or that the supernatant may be subjected to further processing to obtain the intended formulation.

For instance, the supernatant may be concentrated to a lower volume for easier management of the obtained formulation, or it may be dried to obtain a solid formulation according to the invention.

Therefore, in a particular embodiment of the invention, the supernatant separated in step c) of the method for producing a formulation for foliar application in plants is spray-dried to obtain a solid formulation.

In the event that the amount of solids obtained after spray-drying the supernatant obtained in step c) is too low for proper handling, the present invention contemplates the possibility of adding a polymer to the formulation for easier handling thereof. Therefore, in a preferred embodiment of the invention, a polymer is added to the supernatant separated in step c) of the method, prior to spray-drying the supernatant. More preferably, the polymer is maltodextrin. Even more preferably, the maltodextrin is added to the supernatant at a concentration of at least 10% by weight on a dry basis, most preferably, at a concentration of 10% by weight on a dry basis.

It is within the knowledge expected for a person of skill in the art to find proper conditions in which to culture the Trichoderma spp strain as recited in step a) of the method for producing a formulation for foliar application in plants according to the invention so as to obtain a biomass increase sufficient for producing an effective formulation according to the invention.

For instance, the culturing step a) of the method may comprise an initial incubation of the Trichoderma spp strain in a dish to obtain a first stock of spores. The spores thus obtained are cultured in an appropriate broth to finally obtain the desired biomass increase. Culturing the spores in an appropriate broth may also comprise several sub-steps. Each of these sub-steps may be carried out using the same or different culture broths.

In a preferred embodiment, step a) comprises an initial incubation of the Trichoderma spp strain in a dish in a Potato-Dextrose-Agar (PDA) culture medium. Typically, the inoculated dishes are incubated at 28° C. for 5 days.

In another preferred embodiment, culturing the spores in an appropriate broth comprise the sub-steps of: (i) development of a pre-inoculum culture in a first broth; (ii) development of an inoculum culture in the first broth; and (iii) final development of the culture in a reactor in a second broth. The durations and conditions of each sub-step may be adapted according to the equipment and resources available.

The centrifugation of step b) should be carried out in conditions such that the solids contained within the culture medium are properly decanted, so that the liquid phase of the medium may be easily separated from the decanted solids. The optimization of the centrifugation conditions so that such a separation may be achieved is within the expected skill for an expert in the art. In a preferred embodiment, the centrifugation is carried out at a speed of 14000-15000 RPM at room temperature.

In a particularly preferable embodiment, the Trichoderma spp strain is the Trichoderma harzianum strain deposited under Accession Number PTA-125914, the culture of step a) comprises:
I) incubating the Trichoderma spp strain in dishes to obtain a first stock of spores; and
II) culturing the spores obtained in step I), wherein culturing the spores comprises the following sub-steps:
   (i) development of a pre-inoculum culture in a first broth;
   (ii) development of an inoculum culture in the first broth; and
   (iii) final development of the culture in a reactor in a second broth;
      and the centrifugation of step b) is carried out at 14500 rpm at room temperature.

As previously mentioned, that inventors have found that the compounds produced by Trichoderma during its culture have fungicidal and growth-enhancing properties when applied to plants. Therefore, it is yet another aspect of this invention to provide a method for protecting agricultural crop plants against the infection by phytopathogenic fungi and/or promoting growth of said crop plants, comprising applying an effective mount of the formulation of the invention to the plants by means of foliar application.

The method for protecting agricultural crop plants against the infection by phytopathogenic fungi and/or promoting growth of said crop plants of the invention is to be understood as comprising the steps of:
a) culturing a strain of Trichoderma spp in conditions such that an increase of the biomass of the Trichoderma spp strain is observed;
b) centrifuging the culture obtained in step a);
c) separating the solids from the supernatant of the culture medium, thus obtaining a formulation for foliar application in plants from the supernatant of the culture medium; and
d) applying an effective amount of the formulation obtained in step c) to the plants.

By “effective amount” it is to be understood an amount of the formulation which produces a clearly discernible effect of protection against phytopathogenic fungi and/or promotion of growth in the plants the formulation is applied to. The amount will depend, among other factors, on the plants being treated, the climate conditions of the crop site, etc. For example, the formulation may be applied in an amount of about 1.5-3 liters/ha.

Similarly, it is yet another aspect of this invention to provide the use of the formulation for foliar application of the invention for protecting agricultural crop plants against the infection by phytopathogenic fungi and/or promoting growth of said crop plants.

In an embodiment, the crop plants are selected from soybean, wheat, maize, sunflower, cotton, sorghum, alfalfa, flax, canola, chickpea, rice, potato, onion, yerba mate (Ilex paraguariensis), tea. More preferably, the crop plant is soybean.

In another embodiment, the crop plants are fruit crop plants. Preferably, the fruit crop plants are selected from the group consisting of apple tree, pear tree, citrus tree, grapevine, cotton, tomato, cucumber, eggplant, cotton and squash.

In a preferred embodiment of this aspect, the phytopathogenic fungi may be selected, without limitation, from the group consisting of Fusarium sp, Colletotrichum sp., Cercospora sp, Sclerotinia sp., and Rhizoctonia sp.

In a more preferred embodiment of the invention, the phytopathogenic fungi are selected from the group consisting of Fusarium tucumaniae, Colletotrichum truncatum, Cercospora sojina, and Rhizoctonia solani.

In a particularly preferable embodiment, the formulation for foliar application is applied to the plants in combination with at least one chemical pesticidal agent. Preferably, the at least one chemical pesticidal agent is selected from at least one fungicidal agent, at least one non-hormonal herbicidal agent, and at least one hormonal herbicidal agent. More preferably, the at least one fungicidal agent is triazole, strobirulin, or a combination thereof; the at least one non-hormonal herbicidal agent is fomesafen or diclosulam; or the at least one hormonal herbicidal agent is 2,4-dichlorophenoxyacetic acid, picloram, or a combination thereof.

The chemical pesticidal agent may be applied simultaneously with or subsequently to the formulation of the invention.

Additionally, the application of the foliar formulation of the invention may be combined with an inoculation of the seeds prior to sowing them, for maximum effect in the development of the plants. Therefore, the method for protecting agricultural crop plants against the infection by phytopathogenic fungi and/or promoting growth of said crop plants may comprise first inoculating the seeds of the plants with a bioinoculant prior to sowing them. Particularly, the seeds of the plants to be treated with the formulation of the invention may be inoculated with the Trichoderma spp strain grown in the culture medium used to obtain the formulation of the invention. Preferably, the seeds are inoculated with a combination of the Trichoderma spp strain with Bradyrhizobium japonicum.

### EXAMPLES

The invention will now be further described based on the following examples. It is to be understood that these examples are intended for illustrative purposes only, and by no means should be construed to be limiting the scope of the invention, which is only defined by the appended claims.

### Example 1—Preparation of a Formulation According to the Invention

1. Inoculation and Incubation of Initial Dish
A dish culture was performed using Potato-Dextrose-Agar (PDA) as a culture medium. The dish was inoculated by depositing in its center 10 μl of a stock solution of the Trichoderma harzianum strain deposited at the ATCC under Accession Number PTA-125914 preserved at −20° C. The stock solution was composed by a spore suspension made with a 10% glycerol solution.
The dish was incubated in stove at 28° C. during 5 days, in which time the mycelium developed covering the totality of the dish and forming spores.

2. Preparation of Spore Suspension
5 ml of sterile distilled water were added in laminar flow conditions to the culture dish prepared in the previous step, and the spores were retrieved. The obtained suspension was transferred to a sterile Eppendorf flask, and the spore count was determined in the undiluted suspension as well as in 1/10 and 1/100 dilutions with a Neubauer chamber, obtaining a concentration in the order of 10⁹ spores/ml.

3. Development of the Pre-Inoculum Culture
200 ml of Medium 1 (see Table 1) were inoculated with the spores obtained previously to reach an initial concentration of 1×10⁵ spores/ml.

TABLE 1
Composition of Medium 1

| Component | g/L |
|---|---|
| Cane molasses | 30 |
| KNO₃ | 2.52 |
| (NH₄) H₂PO₄ | 2.88 |
| KH₂PO₄ | 0.5 |
| CaCl₂•7H₂0 | 0.3 |
| MgSO₄ | 0.4 |
| FeSO₄ | 0.005 |

The culture was thus incubated during 72 h at 28° C. and 110 RPM.

4. Development of the Inoculum Culture
8 L of Medium 1 were inoculated with the whole pre-inoculum culture obtained in the previous step in a 10 L bottle oxygenated by bubbling at a pressure of 0.4 Kg/cm². The culture was incubated during 48 h at 28° C.

5. Inoculation and Development of the Culture in a Reactor
2000 L of Medium 2 (see Table 2) were inoculated with the entirety of the inoculum culture prepared in the previous step.

TABLE 2
Composition of Medium 2

| Component | g/L |
|---|---|
| Sodium citrate dihydrate | 2.5 |
| KH₂PO₄ | 5 |
| NH₄NO₃ | 2 |
| MgSO₄ | 0.2 |
| CaCl₂•2H₂O | 0.1 |
| Vitamina B6 | 0.005 |
| Trace elements | 0.1 ml (see Table 3) |

TABLE 3
Composition of trace elements solution of Medium 2

| Component | g/L |
|---|---|
| Citric acid | 5 |
| ZnSO₄•7H₂O | 5 |
| Fe (NH₄)₂(SO₄)₂•6H₂O | 1 |
| CuSO₄ | 0.25 |
| MnSO₄ | 0.05 |
| H₃BO₃ | 0.05 |
| Na₂Mo₄•2 H₂O | 0.05 |

The operational variables of the process are described below.
   Operational variables used:
      Initial temperature: 28° C.
      Air flow: 400 L/min≈0.2 vvm
      Air pressure: 0.8 kg/cm²
      Stirring rate: 70 rpm
The culture was maintained in the reactor in the specified conditions during 72 h, and samples were taken every day to monitor the process by determining the pH of the medium, as well as observing the color and density of the culture. Table 4 below provides results expected for the described process, as observed for several experiments. The expected pH values are an average of the values obtained for said experiments.

TABLE 4
Expected results for monitoring the
culture development in the reactor

| Culture time (h) | pH | Color | Density |
|---|---|---|---|
| Post-inoculation | 4.5 | Transparent/caramel | — |
| 24 | 4.9 | Beige | Notable biomass |
| | | | concentration |
| 48 | 5.8 | Brown | High biomass |
| | | | concentration |
| 72 | 6.2-6.8 | Greenish brown | High biomass |
| | | | concentration |

6. Culture Harvest
After 72 h of incubation, the culture was harvested at a flow of 100 L/h and centrifugating at 18000 RPM, storing the supernatant in bladders under sterility conditions, to obtain between 70-80 percent of the total culture volume.

The formulation according to the invention obtained by the procedure described above is herein after referred to as Y-FOLIUM.

### Example 2—Determination of the Fungicidal and Bioenhancer Activity of the Formulation

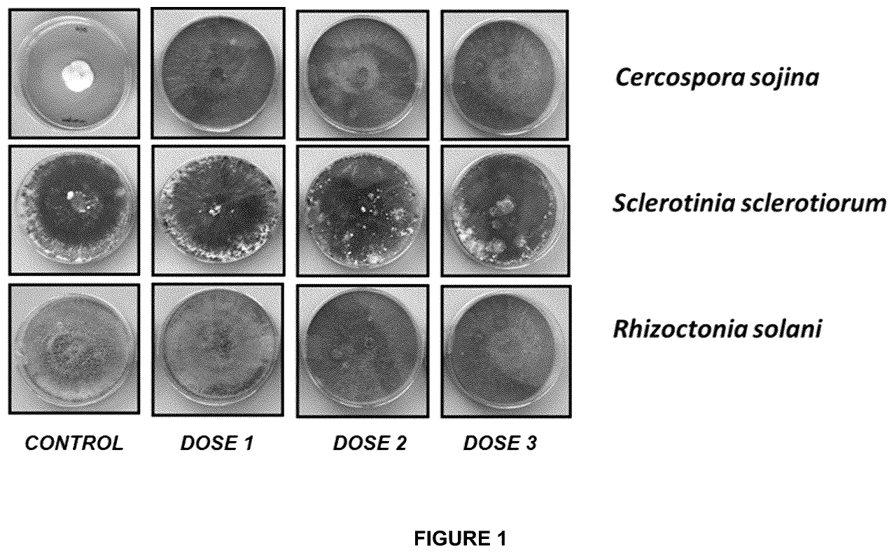

The main pathogens for soybean plants were contacted with different concentrations of the formulation prepared in Example 1. To this end, dilutions of the formulation (40 μl/ml of water and 400 μl/ml of water) were prepared. In said dilutions, as well as in an undiluted sample, the pathogens were diluted to reach a concentration of 10⁵ spores/ml, using sterile distilled water as a control. The obtained mixtures were incubated for 24 hours, and 10 μl of each were then seeded in in the center of a PDA dish. The results can be seen in FIG. 1.

Both in the case of Cercospora sojina and in the case of Rhizoctonia solani, Trichoderma managed to grow over the pathogens and colonize them. In the case of Sclerotinia sclerotiorum the same effect was not observed, but it would not hinder the effectiveness of the formulation since it is a root pathogen, not a foliar one.

To determine the bioenhancer effect of the formulation, the secretion of secondary metabolites in soybean crops was evaluated. Control plants were seeded, which were only inoculated with Bradyrhizobium. When they reached vegetative stage V1 (development of the third trifolium), the formulation was applied to them in 3 different doses (1, 2 and 3 L/hectare).

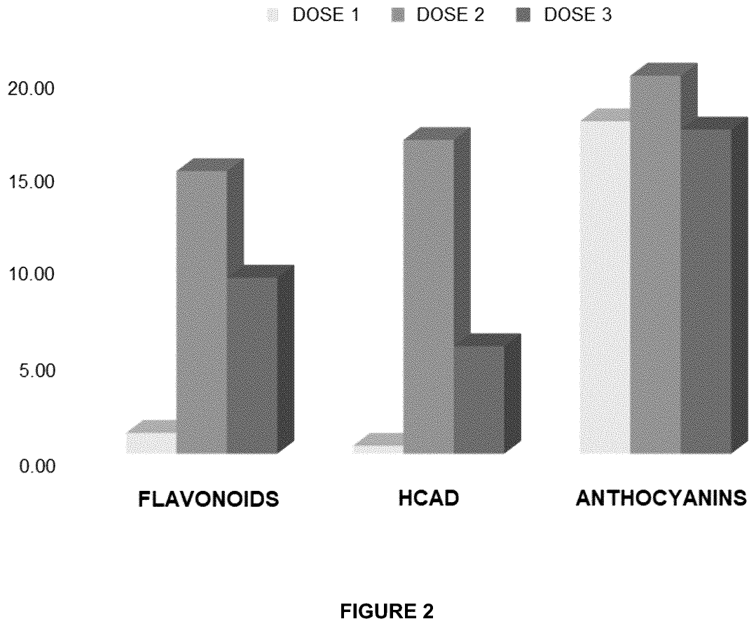

After 48 hours, the content of secondary metabolites was analyzed: anthocyanins, flavonoids and derivates of hydroxycinnamic acid (HCAD), which are markers of the activation of the immune system of plants. Briefly, 0.5 g of the leaves were weighted, frozen with liquid nitrogen, and ground to generate a homogeneous powder to which 3 ml of acidic methanol (1% hydrochloric acid) were added. The obtained mixture was incubated in the dark during 8 h, after which it was agitated and then centrifugated at 3000×g for 2 minutes. The absorbance of the supernatant was measured at 320 nm for determining flavonoids, at 360 nm for determining HCAD, and at 517 nm for determining anthocyanins. The results are shown in FIG. 2.

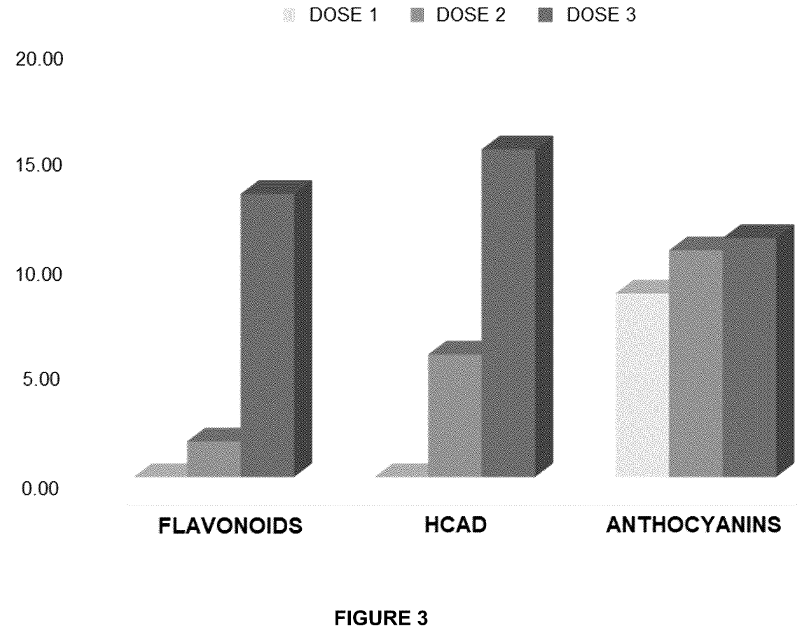

Finally, assays with plants treated with Y-TERRA (bioinoculant comprising the Trichoderma spp strain deposited in the ATCC under Accession Number PTA-125914 and Bradyrhizobium japonicum, described in US 2020/0048157 A1) were performed to evaluate a possible synergistic effect. The treatment of the plants with Y-TERRA was performed according to the product's recommendations. The same doses of the formulation of the invention as used above were applied, and the same metabolites as described above were determined, obtaining the results shown in FIG. 3.

A clear dose/response effect was observed, wherein by increasing the dose of the formulation of the invention, the secretion of every metabolite was increased as well, over the effect already provided by Y-TERRA.

### Example 3—Evaluation of Compatibility and Synergy with Chemical Fungicides

Soybean plants were seeded, both first inoculating the seeds with only Bradyrhizobium and with Y-TERRA, as described above. The inoculations, both with only Bradyrhizobium and with Y-TERRA were performed at a dose rate of 50 mL/kg of seeds. When the plants reached vegetative stage V1, they were treated with a commercial chemical fungicide (Mixtar FI: triazole+strobirulin, applied at 500 mL/ha), the formulation of Example 1 (Y-FOLIUM, applied at 5 L/ha), and a combination of Y-FOLIUM and Mixtar FI (5 L/ha Y-FOLIUM+500 mL/ha Mixtar FI). The assays were repeated three times, performing duplicates of each condition in all cases. 48 hours after foliar application of each treatment, samples of leaves were taken to analyze secondary metabolites as described in Example 2. 10 days after application, two growth parameters (length of the aerial region and length of the radicular region) were determined.

Assays without Y-TERRA

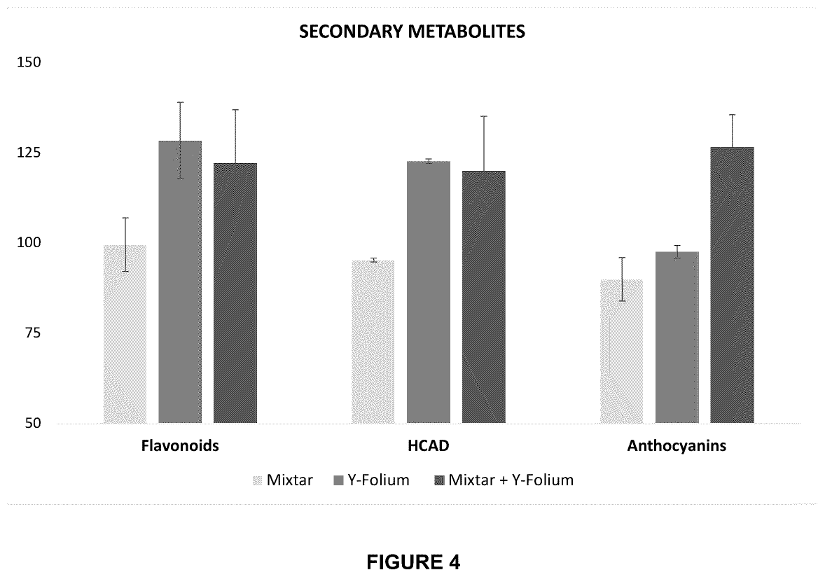

The control plants, wherein no foliar treatment was applied, were considered as 100% of secondary metabolites production. The results are shown in FIG. 4.

In the case of Mixtar FI, it was observed that all values were below 100%, i.e., below those of the control plants. Therefore, applying this product with no pathogen attack has a small negative effect in the development of the plants. In the case of Y-FOLIUM, an increase of about 25% can be observed for both flavonoids and the HCAD. When both products are applied, it can be observed that the formulation of the invention manages to revert the negative effect observed for Mixtar FI, and that the combination produces a synergistic effect for the production of anthocyanins.

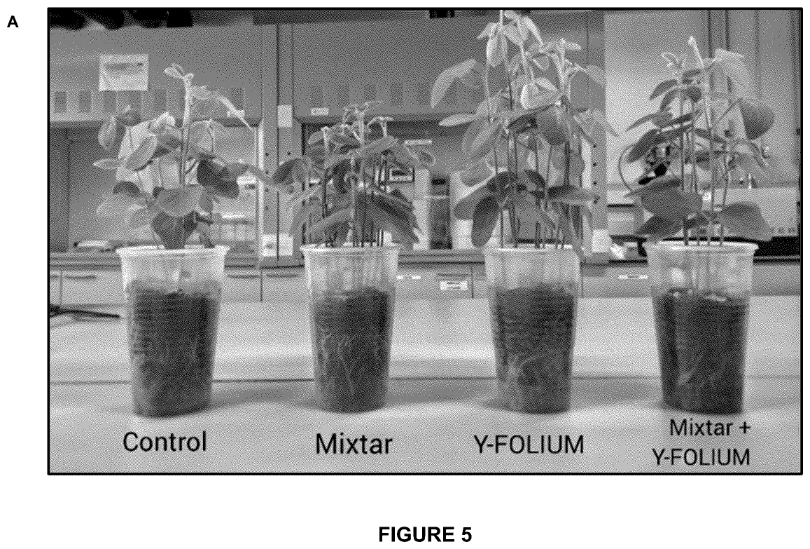

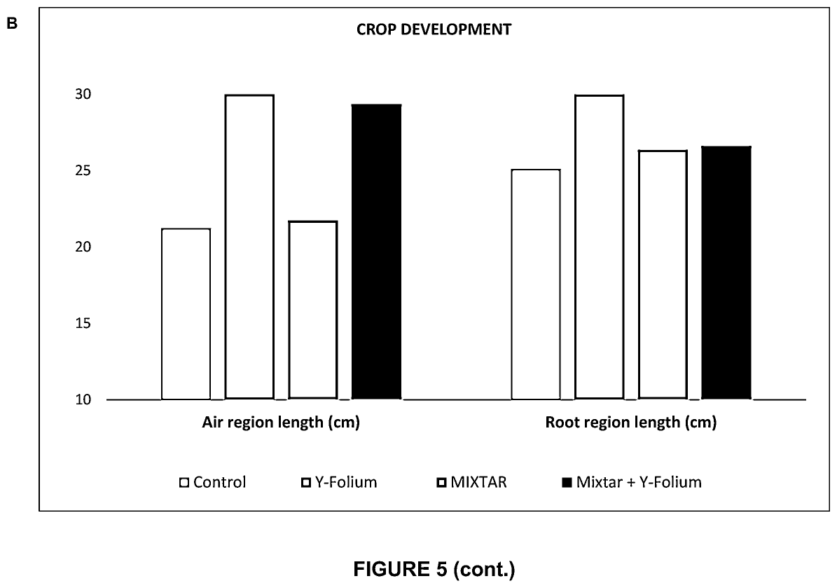

10 days after application the plants were removed from the growth chamber, and their development was analyzed. The results are shown in FIG. 5. It can be seen that Mixtar FI has a slight negative effect in the development, the formulation of the invention had a positive effect, and that the combination does not show the negative effect of Mixtar FI.

Assays with Y-TERRA

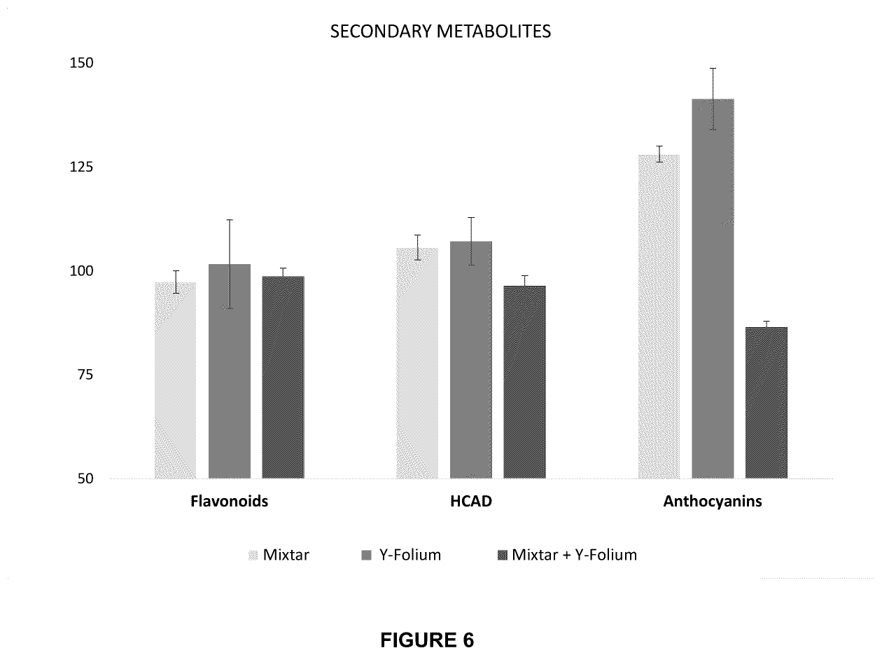

The control plants, wherein no foliar treatment was applied, were considered as 100% of secondary metabolites production. The results are shown in FIG. 6.

In the case of the flavonoids, it can be seen that all three treatments have an average value close to 100%, which implies that the foliar treatment does not produce any effect over the treatment with Y-TERRA (5-10% with respect to the control). The HCAD experienced an increase both with the application of Mixtar FI and Y-FOLIUM, while the combination produced a slight decrease thereof. Finally, the production of anthocyanins was increased over 25% with the application of Mixtar FI and Y-FOLIUM, while the combination produced a significant decrease thereof.

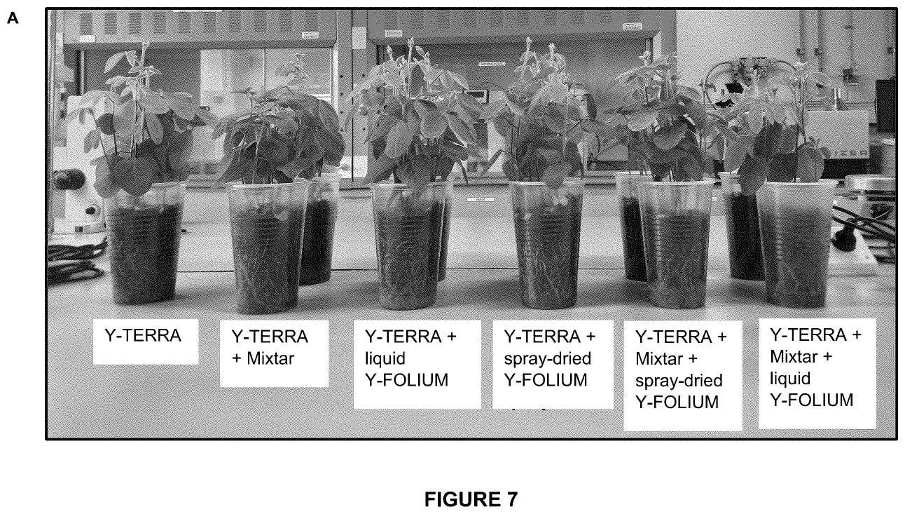

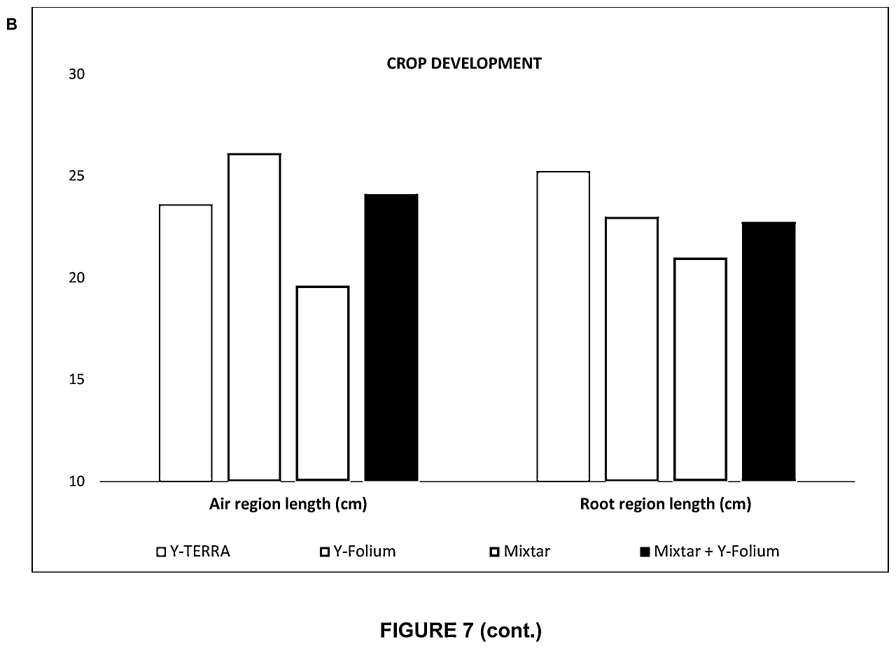

Regarding the development of plants 10 days after application of the treatments, the results can be seen in FIG. 7, including additional tests involving the use of Y-FOLIUM which had been previously spray-dried (250 g/ha). Results analogous to those observed for the assays without Y-TERRA were observed: Mixtar FI has a slight negative effect in the development, Y-FOLIUM had a positive effect, and the combination does not show the negative effect of Mixtar FI.

### Example 4—Field Application Results

Assays were carried out in several locations within the agroecological zones defined by the Argentinean National Service for Health and Agroalimentary Quality (SENASA) for soybean. The defined zones are as follows:
SOUTH: Center, South and West of Buenos Aires and La Pampa.
CENTER: North of Buenos Aires, Santa Fe, Córdoba and Entre Ríos.
ARGENTINEAN NORTHWEST (NOA): Salta, Jujuy, Santiago del Estero, Catamarca and Tucumán; and ARGENTINEAN NORTHEAST (NEA): Corrientes, Chaco, Misiones and Formosa.

The assays selected in this example were carried out in the following locations: Balcarce, Miramar, Barrow (SOUTH), Pergamino and Monte Buey (CENTER).

The product Y-FOLIUM, in two different doses, a chemical fungicide/herbicide agent, a combination of the product Y-FOLIUM with the fungicide/herbicide agent and the commercial product Howler (an elicitor composed of proteins obtained from Acremonium strictum SS71) were applied between R1 and R3. The size of the plots was defined by the agronomist in charge of each trial. The experimental design was a complete block design with 4 randomized replicates, and the applications were carried out with an experimental backpack of carbon dioxide gas.

Table 5 shows the experimental design of the field assays carried out at Pergamino, Barrow and Monte Buey.

TABLE 5
Experimental design of the field assays -
Pergamino, Barrow and Monte Buey

| Treatment | | | | | |
|---|---|---|---|---|---|
| number | Y-FOLIUM | | Fungicide | Howler | Coadjuvant |
| 1 | — | | — | — | — |
| 2 | 1.5 | L/ha | — | — | 200 ml/ha |
| 3 | 3 | L/ha | — | — | 200 ml/ha |
| 4 | — | | — | 2 L/ha | — |
| 5 | — | | Mixtar FI | — | 200 ml/ha |
| 6 | 1.5 | L/ha | Mixtar FI | — | 200 ml/ha |
| 7 | 3 | L/ha | Mixtar FI | — | 200 ml/ha |

Table 6 shows the experimental design of the field assays carried out at Balcarce and Miramar.

TABLE 6
Experimental design of the field assays - Balcarce and Miramar

| Treatment | | | | | |
|---|---|---|---|---|---|
| number | Y-FOLIUM | | Herbicide | Howler | Coadjuvant |
| 1 | — | | — | — | — |
| 2 | 1.5 | L/ha | — | — | 200 ml/ha |
| 3 | 3 | L/ha | — | — | 200 ml/ha |
| 4 | — | | — | 2 L/ha | — |
| 5 | — | | Fomesafen | — | 200 ml/ha |
| 6 | 1.5 | L/ha | Fomesafen | — | 200 ml/ha |
| 7 | 3 | L/ha | Fomesafen | — | 200 ml/ha |

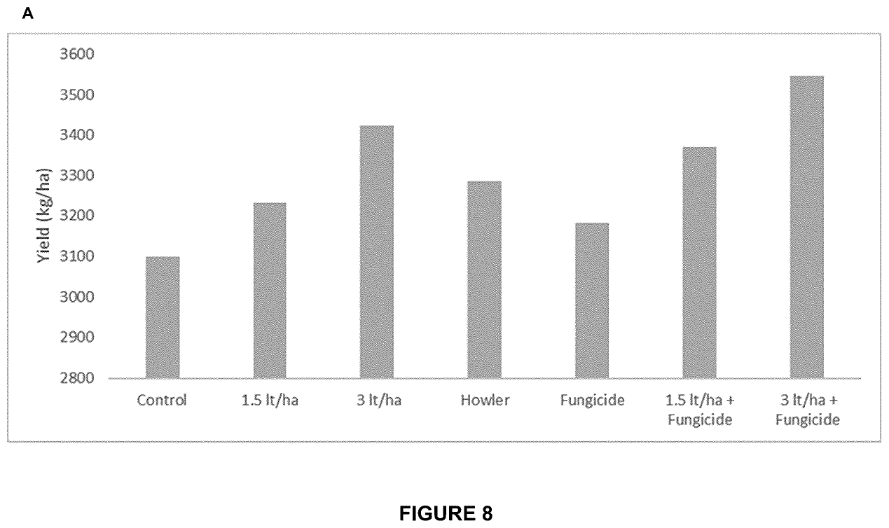

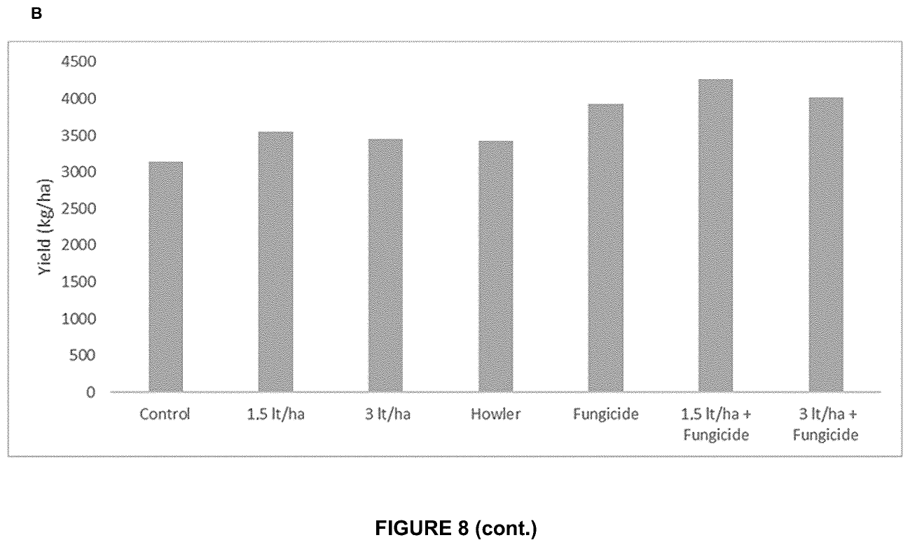

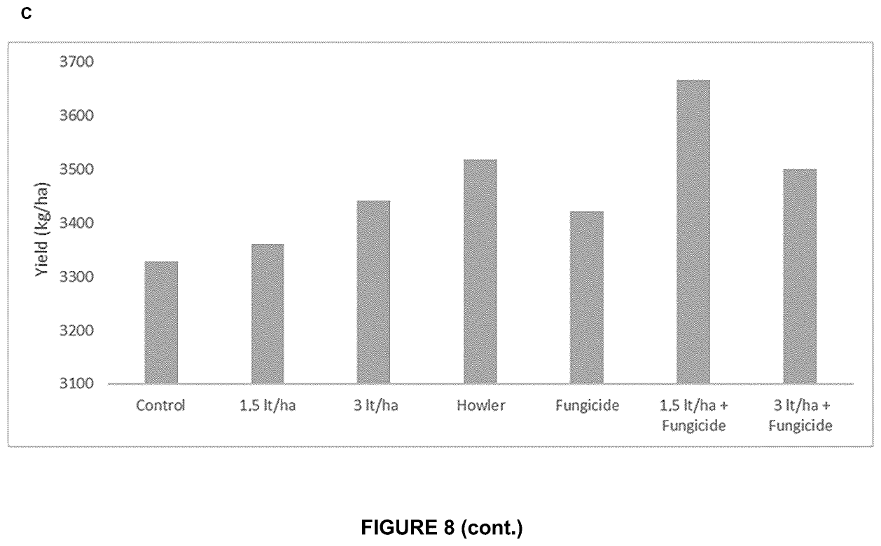

Results: FIG. 8 shows the yields in kg/ha obtained for each treatment, showing positive effects with the doses evaluated in the three locations. Positive effects were also observed after application in combination with the fungicide.

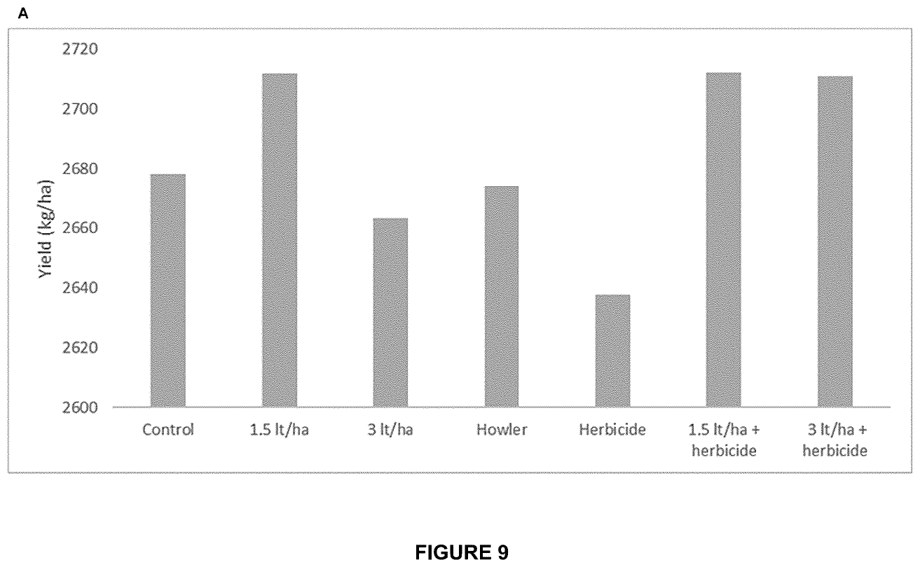

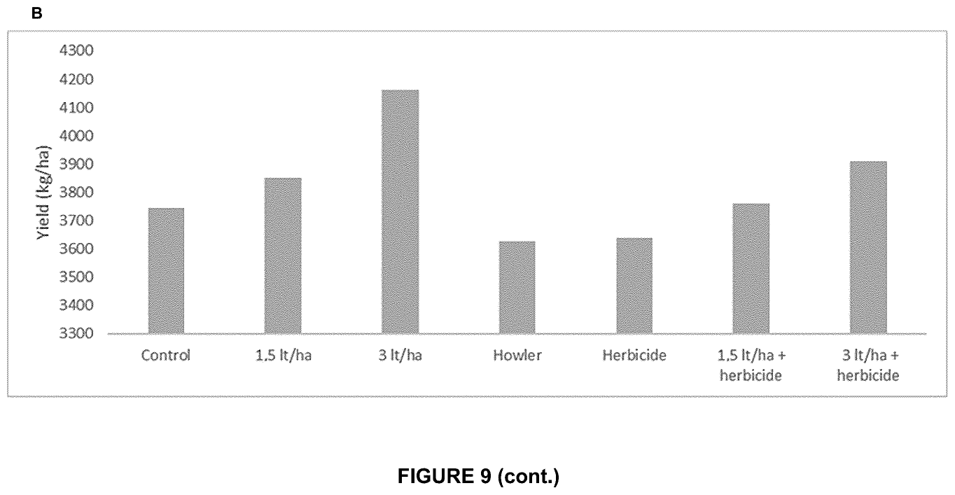

Results: FIG. 9 shows the yields in kg/ha obtained for each treatment, showing positive effects with the doses evaluated in the two locations. Positive effects were also observed after application in combination with the herbicide.

### Example 5—Characterization of a Formulation According to the Invention

A formulation according to the invention was prepared as described in Example 1.

The resulting formulation exhibited a pH of 6.15 and a density of 0.99 g/ml. Additionally, the absence of propagules of Trichoderma harzianum was confirmed by centrifuging a sample of the formulation and determining a concentration of propagules not greater than 3×10 2 propagules/ml.

The formulation was lyophilized, and the proteins present therein were quantified with the Lowry assay. The proteins content determined was 25 μg/ml.

Also, the content of free amino acids in the formulation was determined in an amino acid analyzer using cation exchange chromatography followed by post column derivatization with ninhydrin and measurement at 440 and 570 nm. Prior to the injection of the sample in the amino acid analyzer it was acidified with trichloroacetic acid (TCA) to generate the precipitation of assay interferences. N-Leu was used as an internal standard. The results are shown below in Table 7.

TABLE 7
Free amino acids present in the formulation

| | Cys | Ala | Arg | TOTAL |
|---|---|---|---|---|
| μg aa/100 ml | 22.6 ± 3.0 | 11.1 ± 0.4 | 152.3 ± 0.0 | 186 ± 3.4 |

Additionally, the sample was hydrolyzed with methanesulfonic acid (MSA), and then the resulting amino acids were quantified by an amino acid analyzer with the same conditions and equipment as described above. It is known that certain amino acids exhibit positive effects on the vegetative development, the grain filling, and the biotic and abiotic stress in crops. The results obtained are shown below in Table 7, along with indications on which amino acids are related to the afore mentioned effects.

TABLE 8
Total amino acids determined after hydrolysis of the formulation

| | Lys | Glu | Ile | Phe | Cys | Val | Gly | Ala | Leu | Arg | Pro |
|---|---|---|---|---|---|---|---|---|---|---|---|
| μg aa/100 ml | 14.0 | 22.7 | 27 | 31.5 | 39.8 | 45.6 | 56 | 56.6 | 79.1 | 104.4 | 155.7 |
| Vegetative | ≠ | ≠ | | | | | ≠ | ≠ | | ≠ | ≠ |
| development | | | | | | | | | | | |
| Grain filling | | | | ≠ | | | ≠ | | ≠ | | |
| Biotic stress | | | | ≠ | | | | | | | |
| Abiotic | ≠ | ≠ | ≠ | | ≠ | ≠ | ≠ | ≠ | | ≠ | ≠ |
| stress | | | | | | | | | | | |

Likewise, the quantification of certain phytohormones (zeatin, indole acetic acid and gibberellin GA1) in the sample was performed by high performance liquid chromatography (HPLC) coupled to a triple quadrupole detector (MS-MS). The results are shown below in Table 9.

TABLE 9
Phytohormones determined in the formulation

| | Indole acetic acid | Zeatin | Gibberellin |
|---|---|---|---|
| ng/ml | 9.7 | 54.3 | 11.3 |

### Example 6—Evaluation of Auxin, Cytokinin and Gibberellin Activity

The activity related to auxin, cytokinin and gibberellin in biologic extracts of different plants was determined by bioassays.

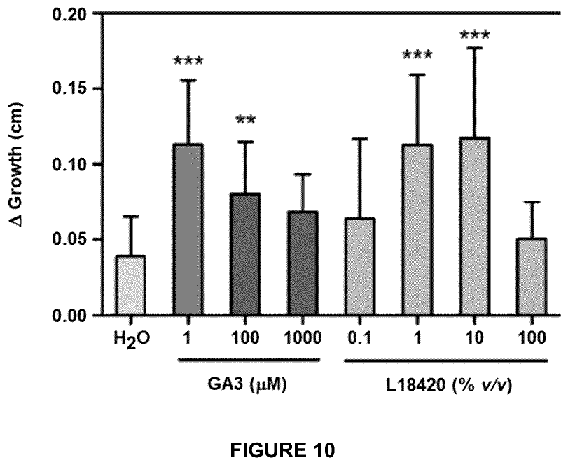

Gibberellin Activity
Gibberellin activity was determined according to the teachings of Silk & Jones (1975) and Salcedo et al. (2020). Briefly, sections of lettuce hypocotyls (Lactuca sativa L., cv. Divina), both controls and samples with different concentrations of the formulation of Example 5 (0.1, 1, 10 and 100% v/v), were incubated for 48 h. As a positive control, gibberellic acid (GA3) was used (concentrations used: 1, 100 and 1000 μM). After the incubation, the length of the hypocotyls was measured. Four independent assays were carried out, with 6 hypocotyls sections per treatment (N=24). The results are shown in FIG. 10.
It can be seen that the treatments with 1 and 10% v/v of the formulation of the invention show a significative increase in the hypocotyl length when compared to the negative control. Said results are similar to the ones obtained with the treatment of 1 μM GA3. Therefore, it is concluded that hormonal activity (gibberellin) was detected in the formulation sample.

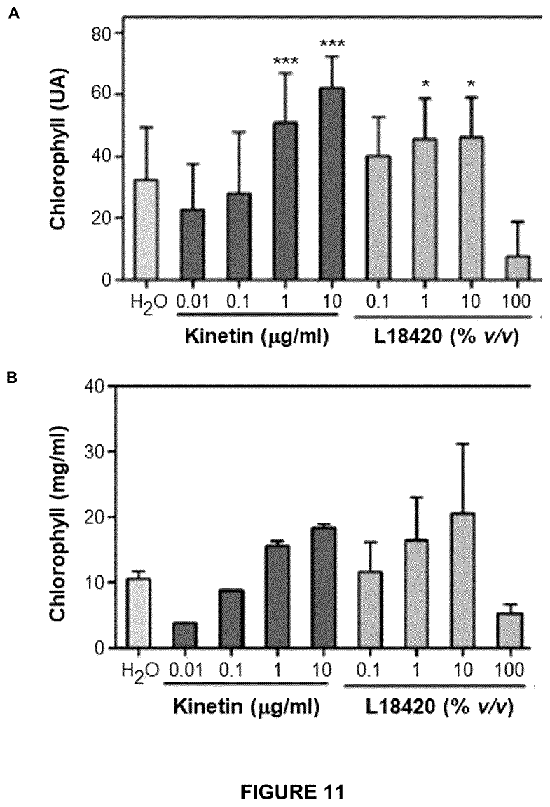

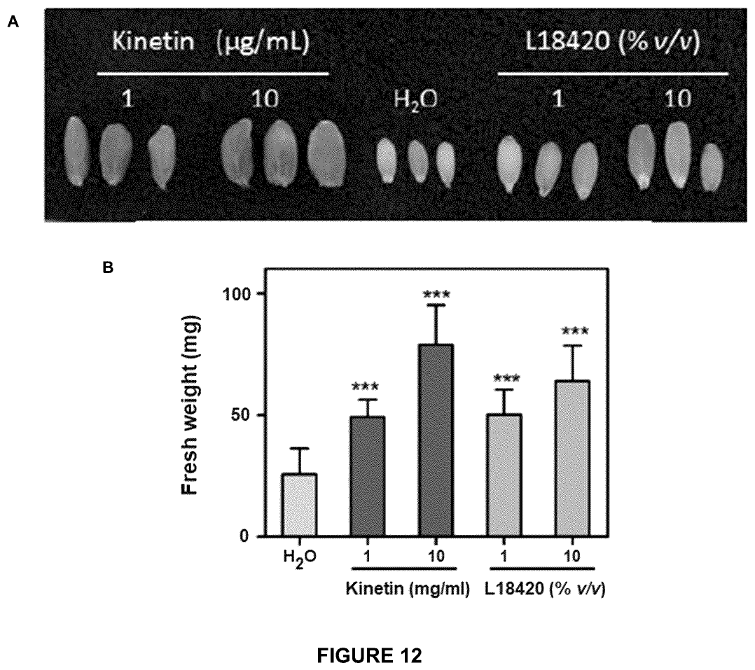

Cytokinin Activity
Cytokinin activity was determined by quantifying the chlorophyll content in cucumber cotyledons (Fletcher et al., 1982). Briefly, cucumber (Cucumis sativus var. Runner) seeds were germinated and grown in darkness for 7 days. Then, the cotyledons were cut and incubated for 20 h under different concentrations of the formulation of Example 5 (0.1, 1, 10 and 100% v/v). As a positive control, different concentrations of a cytokinin analog, kinetin, were used (0.01, 0.1, 1 and 10 μg/ml). Then the cotyledons were exposed to light for 16 hs, and the chlorophyll content was determined by two methods: by means of a manual SPAD detector (Chlorophyll meter SPAD-502, Konica Minolta), and by extracting the chlorophyll with acetone (Arnon 1949). Three independent assays were carried out, with 8 cotyledons per treatment (N=24). The results are shown in FIG. 11.
The cotyledons treated with the formulation showed a statistically significant increase of the greening associated to the presence of hormonal activity. As expected, the reference treatments with kinetin stimulated the greening of the etiolated cotyledons depending on the dose and after being exposed to light.
For the sample of the formulation, the increase of biomass of the cotyledons was also determined according to Narain and Laloraya (1974), proving that the formulation exhibited hormonal activity (FIG. 12).

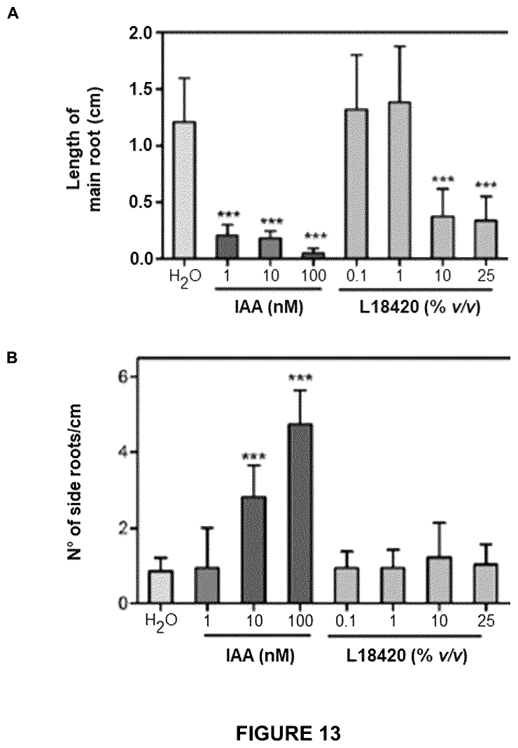

Auxin Activity
Auxin activity was determined by measuring the growth of the main root in seedlings of Arabidopsis thaliana, and the amount of side roots to estimate the root density according to Iglesias et al. (2014). Briefly, 4-days old A. thaliana seedlings were grown in a MS-Agar medium supplemented with different concentrations of the formulation of Example 5 (0.1, 1, 10, 25% v/v). As reference, different concentrations of indole-acetic acid (IAA) were used (1, 10 and 100 nM). Three independent assays were carried out, with 8 seedlings per treatment (N=24). The results are shown in FIG. 13.
As expected, the plants treated with IAA exhibited dose-dependent shortening of the main root and increase in the number of side roots. The seedlings treated with the formulation also exhibited dose-dependent shortening of the main root and slight increase in the number of side roots.

1. A formulation for foliar application in plants comprising an extract obtained from the culture medium of a strain of Trichoderma spp.

2. The formulation of claim 1, wherein the extract is obtained from the culture medium of a strain of Trichoderma harzianum.

3. The formulation of claim 1, wherein the strain of Trichoderma harzianum is the Trichoderma harzianum strain deposited at the ATCC under Accession Number PTA-125914.

4. The formulation of claim 1, wherein the formulation is in liquid form, and comprises 20-30 μg/ml of proteins.

5. The formulation of claim 1, wherein the formulation is in liquid form, and comprises cysteine, alanine, and arginine as free amino acids.

6. The formulation of claim 5, wherein the formulation comprises 15-25 μg/100 ml cysteine, 9-13 μg/100 ml alanine and 140-160 μg/100 ml arginine.

7. The formulation of claim 1, wherein the formulation is in liquid form, and comprises lysine, glutamate, isoleucine, phenylalanine, cysteine, valine, glycine, alanine, leucine, arginine, and proline after being subjected to hydrolysis conditions.

8. The formulation of claim 7, wherein the formulation comprises 12-16 μg/100 ml lysine, 20-25 μg/100 ml glutamate, 25-30 μg/100 ml isoleucine, 28-33 μg/100 ml phenylalanine, 45-55 μg/100 ml cysteine, 40-50 μg/100 ml valine, 50-60 μg/100 ml glycine, 50-60 μg/100 ml alanine, 75-85 μg/100 ml leucine, 100-110 μg/100 ml arginine and 150-160 μg/100 ml proline.

9. The formulation of claim 1, wherein the formulation is in liquid form, and comprises indole acetic acid, zeatin and gibberellin (GA1)

10. The formulation of claim 9, wherein the formulation comprises 9.7 ng/ml indole acetic acid, 54.3 ng/ml zeatin and 11.3 ng/ml GA1.

11. A method for producing a formulation for foliar application in plants, comprising
a) culturing a strain of Trichoderma spp in conditions such that an increase of the biomass of the Trichoderma spp strain is observed;
b) centrifuging the culture obtained in step a); and
c) separating the solids from the supernatant of the culture medium;
thus obtaining the formulation from the supernatant of the culture medium.

12. The method of claim 11, wherein the supernatant separated in step c) is spray-dried to obtain a solid formulation.

13. The method of claim 12, wherein a polymer is added to the supernatant separated in step c) of the method, prior to spray-drying the supernatant.

14. The method of claim 13, wherein the polymer is maltodextrin.

15. The method of claim 14, wherein the maltodextrin is added to the supernatant at a concentration of at least 10% on a dry basis, preferably, at a concentration of 10% on a dry basis.

16. A formulation for foliar application in plants which is obtained by
a) culturing a strain of Trichoderma harzianum in conditions such that an increase of the biomass of the Trichoderma harzianum strain is observed;
b) centrifuging the culture obtained in step a); and
c) separating the solid Trichoderma harzianum biomass from the supernatant of the culture medium;
thus obtaining the formulation from the supernatant of the culture medium.

17. A method for protecting agricultural crop plants against the infection by phytopathogenic fungi and/or promoting growth of said crop plants, comprising applying an effective mount of formulation comprising an extract obtained from the culture medium of a strain of Trichoderma spp to the plants by means of foliar application.

18. The method of claim 17, wherein the crop plants are selected from soybean, wheat, maize, sunflower, cotton, sorghum, alfalfa, flax, canola, chickpea, rice, potato, onion, yerba mate (Ilex paraguariensis), tea.

19. The method of claim 17, wherein the crop plants are fruit crop plants.

20. The method of claim 19, wherein the fruit crop plants are selected from the group consisting of apple tree, pear tree, citrus tree, grapevine, tomato, cucumber, eggplant, cotton and squash.

21. The method of claim 17, wherein the phytopathogenic fungi may be selected, without limitation, from the group consisting of Fusarium sp, Colletotrichum sp., Cercospora sp, Sclerotinia sp., and Rhizoctonia sp.

22. The method of claim 17, wherein the formulation is applied to the plants in combination with at least one chemical pesticidal agent.

23. The method of claim 22, wherein the at least one chemical pesticidal agent selected from at least one fungicide, at least one non-hormonal herbicide, and at least one hormonal herbicide.

24. The method of claim 23, wherein the at least one fungicidal agent is triazole, strobirulin, or a combination thereof.

25. The method of claim 23, wherein the at least at least one non-hormonal herbicidal agent is fomesafen or diclosulam.

26. The method of claim 23, wherein the at least one hormonal herbicidal agent is 2,4-dichlorophenoxyacetic acid, picloram, or a combination thereof.

27. The method of claim 23, wherein the at least one pesticidal agent is a fungicidal agent containing a combination of triazole and strobirulin.

28. Use of a formulation comprising an extract obtained from the culture medium of a strain of Trichoderma spp for protecting agricultural crop plants against the infection by phytopathogenic fungi and/or promoting growth of said crop plants.

29. The use of claim 28, wherein the crop plants are selected from soybean, wheat, maize, sunflower, cotton, sorghum, alfalfa, flax, canola, chickpea, rice, potato, onion, yerba mate (Ilex paraguariensis), tea.

30. The use of claim 28, wherein the crop plants are fruit crop plants.

31. The use of claim 30, wherein the fruit crop plants are selected from the group consisting of apple tree, pear tree, citrus tree, grapevine, tomato, cucumber, eggplant, cotton and squash.

32. The use of claim 28, wherein the phytopathogenic fungi may be selected, without limitation, from the group consisting of Fusarium sp, Colletotrichum sp., Cercospora sp, Sclerotinia sp., and Rhizoctonia sp.

33. The use of claim 28, wherein the phytopathogenic fungi are selected from the group consisting of Fusarium tucumaniae, Colletotrichum truncatum, Cercospora sojina, and Rhizoctonia solani.
